(19) United States
(12) Patent Application Publication
(10) Pub. No.: US 2022/0090140 A1
(43) Pub. Date: Mar. 24, 2022

(54) Efficient Impurity Removal Using A Diafiltration Process

(71) Applicant: Janssen Biotech, Inc., Horsham, PA (US)

(72) Inventors: Hsu-Feng Ko, North Wales, PA (US); Ravinder Bhatia, Blue Bell, PA (US); Soumya Mohanan Krishnathu, Chalfont, PA (US); Vaishali Yannone, Warrington, PA (US); Jeffrey Edward Landau, Lafayette Hill, PA (US); Bas Diepenbroek, Utrecht (NL); Guus Bjorn Erkens, Boskoop (NL); Elisabeth Meulenbroek, Oegstgeest (NL); Feras Nachmi Alazi, Pijnacker (NL)

(21) Appl. No.: 17/595,160

(22) PCT Filed: Apr. 21, 2020
(86) PCT No.: PCT/IB2020/053775

§ 371 Date: Nov. 10, 2021

Related U.S. Application Data
(60) Provisional application No. 62/847,420, filed on May 14, 2019.

(51) Int. Cl. C12N 15/86

(52) U.S. Cl. CPC C12N 15/86; C12N 2710/10351

(57) ABSTRACT

A method for purifying a viral vector from a solution including the viral vector and host cell proteins (HCP) is provided. The method includes circulating the solution across an ultrafiltration/diafiltration membrane using tangential flow filtration (TFF) mode with continuous addition of diafiltration buffer. The method further includes filtering the solution across the ultrafiltration/diafiltration membrane to provide a permeate and a retentate. The retentate is collected such that a purified viral vector solution is obtained and retained in the retentate. A volume of the retentate is kept constant by the continuous addition of diafiltration buffer and the HCP is filtered out via the permeate.

### TECHNICAL FIELD

The disclosure relates to the field of biotechnology and medicine and, more particularly, to the purification of biological products by filtration.

### BACKGROUND

Biological products, such as proteins and viral vectors, ideally contain low levels of chemical impurities. Viral vectors must be purified by removal of host cell protein (HCP) impurities left over from the cell culture. In the area of recombinant viral vectors, for example, there is a need for large-scale manufacture and purification of pharmaceutical-grade viruses. Recombinant adenoviruses are a well-known class of viral vectors for use in gene therapy and for vaccination purposes.

After propagation of the viruses in the cells, it is usually necessary to purify the viruses for use in patients or vaccines. Prior art purification methods include, for example, chromatography and filtration processes. For example, in certain purification processes, a step of ultrafiltration/diafiltration may be used to concentrate the virus and/or to exchange the buffer in which the virus is kept.

However, despite these prior art methods, there is a need for the development of an efficient process for purification of viral vectors which provides for enhanced removal of impurities.

### BRIEF SUMMARY

Provided is a method of purifying a viral vector from a solution comprising the viral vector and impurities, such as HCPs. The method comprises a) circulating the solution across an ultrafiltration/diafiltration membrane using tangential flow filtration (TFF) mode at a loading of between 5 to 100 liters of bioreactor harvest per square meter of surface area of the ultrafiltration/diafiltration membrane and under a pulsatile flow having a frequency of 1.66 to 50 Hz and an amplitude of 2% to 25%, with a continuous addition of diafiltration buffer; b) filtering the solution across the ultrafiltration/diafiltration membrane to provide a permeate and a retentate; and c) collecting the retentate, such that a purified viral vector solution is obtained. A volume of the retentate is kept constant by the continuous addition of diafiltration buffer. The viral vector is retained in the retentate. The HCP is filtered out via the permeate, and a reduction of the HCP from the solution is between 1.5 log and 4.3 log.

### BRIEF DESCRIPTION OF THE DRAWINGS

FIG. 1 is a schematic diagram of an ultrafiltration/diafiltration process according to an embodiment of the present invention.

FIG. 2 depicts an oscillating flow profile for the crossflow for a portion of an ultrafiltration/diafiltration process according to an embodiment of the present invention.

FIG. 3 depicts a steady flow profile for the crossflow for a portion of an ultrafiltration/diafiltration process.

### DETAILED DESCRIPTION

Provided is a method for purifying a viral vector from a solution comprising the viral vector and impurities.

While the following discussion focuses on application of the present invention to the purification of viral vectors, it will be understood that the process may be applicable to a variety of biological materials.

Viruses can be propagated in cells (sometimes referred to as ‘host cells’). Cells are cultured to increase cell and virus numbers and/or virus titers. Culturing a cell is done to enable it to metabolize and produce a virus of interest. This can be accomplished by methods as such well known to persons skilled in the art.

Examples of viral vectors suitable for use with the invention include, but are not limited to adenoviral vectors, adeno-associated virus vectors, pox virus vectors, modified vaccinia ankara (MVA) vectors, enteric virus vectors, Venezuelan Equine Encephalitis virus vectors, Semliki Forest Virus vectors, Tobacco Mosaic Virus vectors, lentiviral vectors, etc.

In certain embodiments of the invention, the vector is an adenovirus vector. An adenovirus according to the invention belongs to the family of the Adenoviridae, and preferably is one that belongs to the genus Mastadenovirus. It can be a human adenovirus, but also an adenovirus that infects other species, including but not limited to a bovine adenovirus (e.g. bovine adenovirus 3, BAdV3), a canine adenovirus (e.g. CAdV2), a porcine adenovirus (e.g. PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV). In the invention, a human adenovirus is meant if referred to as Ad without indication of species, e.g. the brief notation “Ad26” means the same as HadV26, which is human adenovirus serotype 26. Also as used herein, the notation “rAd” means recombinant adenovirus, e.g., “rAd26” refers to recombinant human adenovirus 26.

In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like.

In certain embodiments the recombinant adenovirus vector useful in the invention is derived mainly or entirely from Ad26 (i.e., the vector is rAd26). The preparation of recombinant adenoviral vectors is well known in the art. Preparation of rAd26 vectors is described, for example, in WO 2007/104792 and in Abbink et al., (2007) Virol 81(9): 4654-63. Exemplary genome sequences of Ad26 are found in GenBank Accession EF 153474 and in SEQ ID NO:1 of WO 2007/104792. Examples of vectors useful for the invention for instance include those described in WO 2012/082918, the disclosure of which is incorporated herein by reference in its entirety.

However, it will be understood that the method of the present invention is not limited to adenoviruses viruses, but rather may be applicable to a broad range of other viruses (e.g., adeno associated virus, pox viruses, iridoviruses, herpes viruses, papovaviruses, paramyxoviruses, orthomyxoviruses, retroviruses, vaccinia virus, rotaviruses, flaviviruses) and other biologic materials, such as proteins.

Biological products typically include a variety of contaminants or impurities remaining from the cell culture. A “contaminant” or “impurity” is any component of the new drug product that is not the drug substance or an excipient in the drug product. The inventive process is targeted to removal of host cell proteins (HCP), but other impurities may or may not be removed in conjunction with the removal of HCPs. Examples of such impurities include, but are not limited to, host cell DNA (HC-DNA), Triton X-100, Tris, sodium phosphate (monobasic and dibasic), magnesium chloride (MgCl₂), HEPES and insulin.

More particularly, the virus (product) is released into the media after chemical lysis of the cell membrane and then impurities in the lyzed harvest material are flocculated. After removal of these impurities, the material is clarified to load on to a chromatographic membrane. The resulting material (viral vector) is a concentrated product which also includes other impurities, such as HCP or HC-DNA.

According to the present invention, the viral vector is then subjected to ultrafiltration/diafiltration for removal of the impurities, such as HCP left over from the cell culture, for purification of the viral vector. A preferred ultrafiltration/diafiltration process is tangential flow filtration.

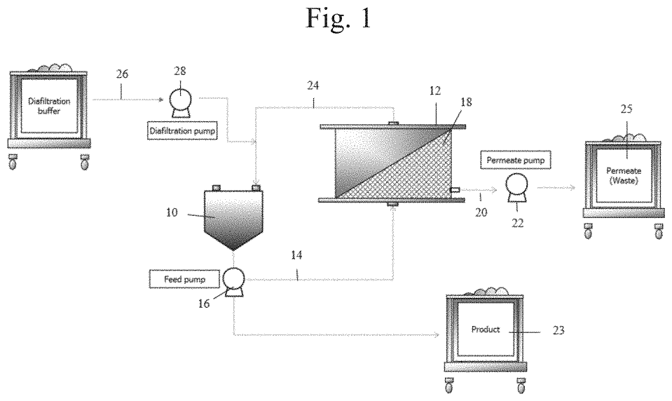

Referring to FIG. 1, there is provided a schematic diagram of an ultrafiltration/diafiltration process in accordance with an embodiment of the present invention. A feed tank 10 comprises the sample solution to be filtered, for example a solution containing the viral vector of interest. The solution enters the filtration unit 12 through a feed channel or feed line 14. Preferably, a first mechanical pump 16 is provided in the feed line 14 for circulating and controlling the solution flow. The filtration unit 12 comprises an ultrafiltration/diafiltration membrane 18. As the feed solution is supplied to the filtration unit 12, the ultrafiltration/diafiltration membrane 18 separates the solution into a permeate and a retentate.

Diafiltration buffer is continuously added to the feed solution in the feed tank 10 via a diafiltration line 26, in order to maintain the overall product (retentate) volume. A second pump 28 may be provided in the diafiltration buffer line 26 to control the supply of the diafiltration buffer to the feed tank 10. Any known buffer that would not affect the virus to be purified may be utilized. Preferably, the buffer has a pH of approximately 6.2 and contains small molecules to stabilize the product/virus particles. Preferably, between 7 and 11 diafiltration volumes (DFVs) are exchanged during the diafiltration step. More preferably, 10 DFVs are exchanged during the diafiltration step.

In one embodiment, one or more detectors (not shown) may be provided in the feed line 14 for measuring the pressure across the ultrafiltration/diafiltration membrane 18.

A pressure differential across the ultrafiltration/diafiltration membrane 18 causes the feed solution, and more particularly the impurities, to flow through the ultrafiltration/diafiltration membrane 18, such that the impurities are contained in the permeate. More particularly, the feed solution containing the viral vector is passed across the ultrafiltration/diafiltration membrane 18, such that impurities are removed from the feed solution and retained in the permeate, while the viral vector is unable to pass through the ultrafiltration/diafiltration membrane 18 and is thereby retained in the retentate.

The surface area of the ultrafiltration/diafiltration membrane 18 may be selected depending upon the volume of feed solution to be purified. The ultrafiltration/diafiltration membrane 18 may have different pore sizes depending on the biological material (e.g., viral vector) being purified and the impurities contained therein. Preferably, the ultrafiltration/diafiltration membrane 18 has a pore size sufficiently small to retain the viral vector in the retentate, but large enough to effectively clear impurities (i.e., to allow the impurities to pass through the membrane pores) in the permeate. For adenovirus vector, the ultrafiltration/diafiltration process utilizes a membrane 18 having a Nominal Molecular Weight Limit (NMWL) in the range of from 100 to 1,000 kilodaltons (kDa), preferably in the range of from 300 to 500 kDa, and more preferably 300 kDa. As such, impurities, such as HCPs (molecular mass of approximately 10 to 200 kDa), are able to pass through the ultrafiltration/diafiltration membrane 18 (and be included in the permeate), while the viral particles, which are larger than the pores, are retained by the ultrafiltration/diafiltration membrane 18 in the retentate. That is, the retentate contains the end product (virus)

The ultrafiltration/diafiltration membrane 18 may be comprised of, for example, regenerated cellulose, polyethersulfone, polysulfone, or derivatives thereof. The ultrafiltration/diafiltration membrane 18 may be of any known type or configuration, for example, a flat sheet or plate, a spiral wound member, a tubular member, or hollow fibers. In one embodiment of the present invention, the ultrafiltration/diafiltration membrane 18 is a Pellicon® 2 Ultrafiltration Cassette, manufactured by MilliporeSigma.

The permeate exits the filtration unit 12 through a permeate channel or permeate line 20 and is sent to a permeate collection tank 25. In one embodiment, as shown in FIG. 1, a third mechanical pump 22 is provided in the permeate line 20 for controlling the flow of the permeate through the permeate line 20. That is, the separation of impurities from the viral vector is aided by the first pump 16, which feeds and recirculates the feed solution and retentate, and the third pump 22, which facilitates passage of the impurities (e.g., HCP) through the membrane pores and removal of the permeate.

The retentate, which comprises the viral vector of interest, passes into a retentate channel or retentate line 24, which is recirculated back into the feed tank 10. The first pump 16 supplies and recirculates the feed solution/retentate to and across the ultrafiltration/diafiltration membrane 18 at a flux of approximately 250 liters/m²/hour (LMH) to approximately 400 LMH, and more preferably approximately 360 LMH. Preferably, the feed solution/retentate is maintained at a constant flow rate and volume. The feed solution/retentate is preferably supplied to the ultrafiltration/diafiltration membrane 18 at a loading of between 5 to 100 L bioreactor harvest per m² of membrane area, more preferably 5 to 60 L bioreactor harvest per m² of membrane area, and most preferably a loading of between 5 and 45 L bioreactor harvest per m² of membrane area.

Operation of the third pump 22 and the flow rate of the permeate is a function of (i.e., dependent upon) operation of the first pump 16 and flow rate of the feed solution/retentate. Preferably, the flowrate of the permeate is set to be less than 20% of the feed solution/retentate, more preferably between 5% and 15%, and most preferably approximately 10%. Thus, where the feed solution/retentate is maintained at a flowrate of between 250 and 400 LMH, the permeate is preferably maintained by the third pump 22 at a flowrate between 25 and 40 LMH, and most preferably at a flowrate of 36 LMH (i.e., 10% of the target flow setpoint of 360 LMH of the feed solution/retentate).

In one embodiment, the first pump 16 is preferably a positive displacement pump. In one embodiment, both the first pump 16 and the third pump 22 are positive displacement pumps. Examples of positive displacement pumps that may be utilized include, for example, a rotary lobe pump, a progressive cavity pump, a rotary gear pump, a piston pump, a diaphragm pump, a screw pump, a gear pump, a hydraulic pump, a rotary vane pump, a peristaltic pump, a rope pump and a flexible impeller pump. In a preferred embodiment, the first pump 16 is a peristaltic pump. Preferably, the third pump 22 is also a peristaltic pump.

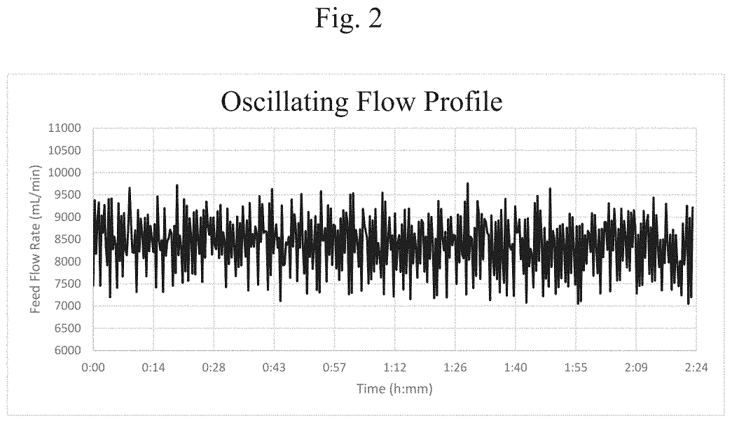

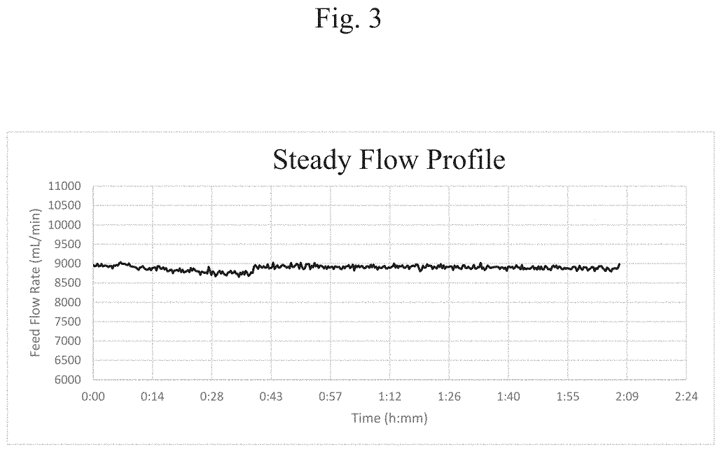

In a preferred embodiment, the ultrafiltration/diafiltration process is carried out under an oscillating flow profile, where the oscillating flow profile results in a pulsating fluid action. Preferably, only the first pump 16 is operating under an oscillating flow profile, while the other pumps in the system are operating under a relatively steady (non-oscillating) flow profile, meaning that the flow profile may exhibit oscillations of small amplitudes but is relatively steady. However, it is also possible that other pumps, such as the third pump 22, are also operating under an oscillating flow profile. For example, a preferred oscillating flow profile of the process is shown in FIG. 2, as compared with a smoother, steady flow profile as shown in FIG. 3.

The pulsating fluid action caused by the flow oscillations, as shown in FIG. 2, enables a larger amount of impurities (e.g., HCPs) to pass through the ultrafiltration/diafiltration membrane 18 into the permeate, than would be enabled by a steadier fluid action as would be achieved by the smoother, steady flow profile of FIG. 3. Preferably, there is a greater than 1.5 log impurities (e.g., HCP) reduction by the ultrafiltration/diafiltration process of the present invention, and more preferably between 1.5 and 4.3 log impurities reduction, and most preferably between 1.5 and 2.3 log impurities reduction.

In a preferred embodiment, the first pump 16 is preferably operated to achieve a pulsatile flow of a predetermined frequency and amplitude. More preferably, the pulsatile flow of the first pump 16 has a frequency of 1.66 to 50 Hz, more preferably of 1.66 to 33 Hz, and even more preferably of 1.66 to 25 Hz. Preferably, the pulsatile flow of the first pump 16 has a corresponding amplitude of 2% to 25%.

In particular, by conducting the ultrafiltration/diafiltration process under the condition of pulsating fluid action having a frequency of 1.66 to 50 Hz and an amplitude of 2% to 25%, a reduction of greater than 1.5 log, and more particularly a reduction of from 1.5 to 4.3 log, in the impurities (e.g., HCP) can be achieved. This is significantly greater than the reduction that would be achieved by conventional processes.

In one embodiment, the first pump 16 is also preferably operated to achieve a predetermined or target volume displacement. More preferably, the first pump 16 is operated to achieve a predetermined or target normalized displacement, expressed in terms of volume displaced per revolution per square meters of the surface area of the ultrafiltration/diafiltration membrane 18 (ml/rev/m²). In one embodiment according to the present invention, the first pump 16 is operated to achieve a normalized displacement in the range of from 10 to 100 mL/rev/m², and preferably in the range of from 17 to 83 mL/rev/m², which yields superior impurity clearance.

The ultrafiltration/diafiltration method according to embodiments of the present invention is exemplified by the following, non-limiting examples.

### INVENTIVE EXAMPLES 1-10

Adenovirus 26 viral vector Anion Exchange (AEX) chromatography eluate was to be processed. The eluate was broken up into manageable batches, and each batch of the eluate was recirculated across a 300 kDa ultrafiltration/diafiltration membrane 18 at a constant flow rate of 360 LMH under an oscillating flow profile (i.e., pulsating fluid action) and a loading of between 30 and 40 L bioreactor harvest per m² of membrane area. The permeate flow rate was maintained at 36 LMH. The frequency of the pulsating fluid action was in the range of 1.66 to 50 Hz and the amplitude thereof was in the range of 2% to 25%. Also, a normalized displacement in the range of from 17 to 83 milliliters per revolution per square meter of surface area of the ultrafiltration/diafiltration membrane was maintained. During filtering of the eluate by the ultrafiltration/diafiltration membrane 18, viral particles were retained in the retentate, while HCPs and other impurities were filtered out via the permeate. During the filtration process, buffer was added to the retentate to maintain a target overall product volume. The ultrafiltration/diafiltration process was complete after 10 DFVs were exchanged. This process was carried out multiple times using AEX eluate having different starting HCP concentrations.

### COMPARATIVE EXAMPLES 1-12

Adenovirus 26 viral vector Anion Exchange (AEX) chromatography eluate was recirculated across a 300 kDa ultrafiltration/diafiltration membrane 18 under a steady flow profile. During filtering of the eluate by the ultrafiltration/diafiltration membrane 18, viral particles were retained in the retentate, while HCPs and other impurities were filtered out via the permeate. Buffer was added to the retentate to maintain a target overall product volume. The ultrafiltration/diafiltration process was complete after 10 DFVs were exchanged. This process was carried out multiple times using AEX eluate having different starting HCP concentrations, different recirculation flow rates, different permeate flow rates and different retentate pressures.

Table 1 provides the results of these various experiments.

TABLE 1
HCP Clearance by Ultrafiltration/Diafiltration

| | | Starting HCP | Ending HCP | |
|---|---|---|---|---|
| | Flow | concentration | concentration | Log |
| Example | Profile | (μg/mL) | (μg/mL) | Reduction |
| Inventive Example 1 | Oscillating | 32.0 | <0.2 | >2.2 |
| Inventive Example 2 | Oscillating | 40.2 | <0.2 | >2.2 |
| Inventive Example 3 | Oscillating | 9.6 | <0.2 | >1.9 |
| Inventive Example 4 | Oscillating | 6.7 | <0.2 | >1.6 |
| Inventive Example 5 | Oscillating | 5.99 | <0.2 | >1.5 |
| Inventive Example 6 | Oscillating | 32.98 | 0.63 | 1.8 |
| Inventive Example 7 | Oscillating | 26.54 | <0.2 | >2.1 |
| Inventive Example 8 | Oscillating | 23.28 | <0.2 | >2.0 |
| Inventive Example 9 | Oscillating | 10.72 | 0.28 | 1.5 |
| Inventive Example 10 | Oscillating | 28.31 | <0.2 | >2.2 |
| Comparative Example 1 | Steady | 37.5 | 5.3 | 1.1 |
| Comparative Example 2 | Steady | 37.5 | 5.2 | 1.2 |
| Comparative Example 3 | Steady | 32.0 | 1.8 | 1.3 |
| Comparative Example 4 | Steady | 37.5 | 2.3 | 1.2 |
| Comparative Example 5 | Steady | 30.6 | 6.0 | 0.71 |
| Comparative Example 6 | Steady | 10.27 | 0.96 | 1.0 |
| Comparative Example 7 | Steady | 10.44 | 1.61 | 0.78 |
| Comparative Example 8 | Steady | 10.2 | 0.59 | 1.2 |
| Comparative Example 9 | Steady | 4.4 | 0.20 | 1.3 |
| Comparative Example 10 | Steady | 10.32 | 0.47 | 1.3 |

In the comparative examples summarized in Table 1, various process parameters, such as the recirculation flow rate, permeate flow rate and retentate pressure, were varied, while maintaining a steady flow profile. Even with the variation of these other process parameters, an effective removal of HCPs (i.e., greater than 1.5 log reduction in HCPs and ending HCP level of less than 0.2 μg/mL) could still not be achieved. As shown in Table 1, only when the ultrafiltration/diafiltration process is carried out under an oscillating flow profile, and a greater than 1.5 log reduction (and more particularly a 1.5 to 4.3 log reduction) in HCPs can be achieved. Presumably, the pulsating fluid mechanism causes the gel layer lining the ultrafiltration/diafiltration membrane 18 to be disturbed to a sufficient extent to allow HCPs to pass through the membrane pores more readily than when the gel layer remains wholly intact under non-pulsating fluid action.

Further experiments were conducted under the conditions of Inventive Examples 1-3, where some examples fell within the preferred range of the frequency and amplitude of the pulsatile flow, while others were outside of the preferred range. These results are summarized in Table 2.

TABLE 2
Parameters of Ultrafiltration/Diafiltration Process

| | | | Frequency | Amplitude | | | | | |
|---|---|---|---|---|---|---|---|---|---|
| | | | (rpm*number | (% of | | Tubing | Displacement | Normalized | Log |
| Run | | | of rollers)/ | flow rate | Membrane | ID | per revolution | Displacement | Reduction |
| No. | # Rollers | RPM* | 60 (Hz) | variation) | Area (m²) | (inch) | (mL/rev) | (mL/rev/m²) | in HCP |
| 1 | 2 | 90 | 3 | 4% | 5 | 1 | 326.7 | 65.2 | >2.21 |
| 2 | 2 | 90 | 3 | 4% | 5 | 1 | 326.7 | 65.2 | >1.64 |
| 3 | 2 | 216 | 7.2 | 23% | 1.5 | 0.5 | 41.5 | 27.67 | >1.7 |
| 4 | 2 | 245.2 | 8.2 | 24% | 1.5 | 0.5 | 41.5 | 27.67 | >1.5 |
| 5 | 1 (4 | 948.5 | 63.2 | 1.90% | 1.5 | 0.5 | 9.7 | 6.5 | 0.37 |
| | pistons) | | | | | | | | |
| 6 | 1 (4 | 1015 | 67.7 | 1.50% | 1.5 | 0.5 | 9.4 | 6.3 | 0.09 |
| | pistons) | | | | | | | | |
| 7 | 1 (4 | 989 | 65.9 | 1.60% | 1.5 | 0.5 | 9.0 | 6.0 | 0.75 |
| | pistons) | | | | | | | | |
| 8 | 1 (4 | 1020 | 68 | 1.70% | 1.5 | 0.5 | 9.2 | 6.1 | 1.3 |
| | pistons) | | | | | | | | |
| 9 | 1 (4 | 1020 | 68 | 2.10% | 1.5 | 0.5 | 9.3 | 6.2 | 1.24 |
| | pistons) | | | | | | | | |

*All RPM setpoints selected to result in 360 LMH retentate flow/flux.

As can be seen by the results summarized in Table 2, where the frequency and amplitude of the pulsatile flow are maintained in the preferred ranges (i.e., frequency of 1.66 to 50 Hz and amplitude of 2% to 25%), there is between 1.5 and 4.3 log impurities reduction. On the other hand, where the frequency and/or amplitude fall outside of the preferred ranges, such as in Run numbers 5-9, the reduction in impurities is significantly lower.

According to the process of the present invention, no further purification is required after the ultrafiltration/diafiltration process. However, it will be understood that the product may optionally be further purified by methods generally known to persons skilled in the art, such as density gradient centrifugation, chromatography and the like.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for purifying a viral vector from a solution comprising the viral vector and host cell proteins (HCP), the method comprising:
a) circulating the solution across an ultrafiltration/diafiltration membrane using tangential flow filtration (TFF) mode at a loading of between 5 and 100 liters of bioreactor harvest per square meter of surface area of the ultrafiltration/diafiltration membrane and under a pulsatile flow having a frequency of 1.66 to 50 Hz and an amplitude of 2% to 25%, with a continuous addition of diafiltration buffer;
b) filtering the solution across the ultrafiltration/diafiltration membrane to provide a permeate and a retentate, a volume of the retentate being kept constant by the continuous addition of diafiltration buffer, the viral vector being retained in the retentate and the HCP being filtered out via the permeate, a reduction of the HCP from the solution being between 1.5 and 4.3 log; and
c) collecting the retentate, such that a purified viral vector solution is obtained.

2. The method of claim 1, wherein viral vector is an adenoviral vector.

3. The method of claim 1, wherein the ultrafiltration/diafiltration membrane has a NMWL of from about 100 kDa to about 500 kDa.

4. The method of claim 3, wherein the ultrafiltration/diafiltration membrane has a NMWL of about 300 kDa.

5. The method of claim 1, wherein a flow rate of the solution to be filtered is in the range of from 250 liters/m²/hour (LMH) to 400 LMH.

6. The method of claim 5, wherein the flow rate of the solution to be filtered is approximately 360 LMH.

7. The method of claim 5, wherein the flow rate of the solution to be filtered is constant.

8. The method of claim 5, wherein a flow rate of the permeate is between 5% and 15% of the flow rate of the solution to be filtered.

9. The method according to claim 1, wherein the filtering of the solution is carried out under using a peristaltic pump.
